(19) United States
(12) Patent Application Publication
(10) Pub. No.: US 2006/0275848 A1
(43) Pub. Date: Dec. 7, 2006

(54) Diagnosis and treatment of microbacterial infections

(75) Inventors: Adriaan P. Koets, Houten (NL); Victor Picrre Maric Gerard Rutten, Bunnik (NL); Willen van Eden, Bilthoven (NL)

Correspondence Address: TRASK BRITT, P.O. BOX 2550, SALT LAKE CITY, UT, 84110, US

(73) Assignee: Universiteit Utrecht Holding B. V., Utrecht (NL)

(21) Appl. No.: 11/363,561

(22) Filed: Feb. 27, 2006

Related U.S. Application Data
(63) Continuation of application No. PCT/NL04/00600, filed on Aug. 27, 2004.

(30) Foreign Application Priority Data
Aug. 27, 2003 (EP) 03077682.7

(51) Int. Cl. G01N 33/554; G01N 33/569

(52) U.S. Cl. 435/7.32

(57) ABSTRACT

The invention provides a method for detecting infection of an animal with a microorganism that causes a slow progressive disease, comprising providing the animal with a protein of the microorganism, or a functional part, derivative and/or analogue thereof, and measuring an immune response of the animal directed against the microorganism. With a method of the invention, diagnosis during early stages of an infection has become possible. In one aspect, the microorganism comprises Mycobacterium avium subspecies paratuberculosis. Preferably, the protein comprises a surface-associated protein. Peptides comprising B-cell epitopes of M. avium ssp paratuberculosis heat shock protein 70, nucleic acid molecules encoding such peptides and diagnostic kits comprising a peptide and/or nucleic acid molecule of the invention are also herewith provided.

### CROSS-REFERENCE TO RELATED APPLICATIONS

This application is a continuation of PCT International Patent Application No. PCT/NL2004/000600, filed on Aug. 27, 2004, designating the United States of America, and published, in English, as PCT International Publication No. WO 2005/022160 A2 on Mar. 10, 2005, which application claims priority to European Patent Application Serial No. 03077682.7 filed Aug. 27, 2003, the entire contents of each of which are hereby incorporated herein by this reference.

### TECHNICAL FIELD

The invention relates to the field of immunology. More specifically, the invention relates to diagnosis and treatment of slow progressive infectious diseases.

### BACKGROUND

Complex cellular organisms such as mammals are often challenged by pathogenic microorganisms. Despite many promising medical developments during the last decades, infections of man and animals, such as cattle and pets, still have substantial impact on human as well as animal welfare, healthcare and food production costs. In order to provide adequate treatment, early diagnosis of infection is of utmost importance. However, a significant number of microorganisms cause slow progressive infections that are barely, if at all, detectable in an early stage. Such latency periods often last for years. A prominent group of microorganisms causing slow progressive infections is, for instance, that of the Mycobacteria, e.g., M. avium, M. avium ssp. paratuberculosis, M. tuberculosis, and M. leprae.

Pathogenic mycobacteria are a major cause of disease and mortality in humans as well as in many other mammalian species. In humans, tuberculosis and leprosy are major diseases worldwide, with tuberculosis alone causing over three million deaths each year. Similarly, tuberculosis and paratuberculosis are diseases affecting, among others, economically important food-producing species such as cattle. Apart from the direct economical damage at the producer's level, both diseases also pose a zoonotic risk as transmission of mycobacteria via contaminated food products to humans is possible. Despite major efforts, both accurate and timely diagnosis remains cumbersome and the possibilities of protecting susceptible individuals through vaccination strategies remain limited.

Most of these pathogenic mycobacteria share pathogenicity mechanisms that allow them to effectively hide from the immune system and evade a number of immune-mediated mechanisms. They share properties that enable prolonged latency periods during which they defy host-derived immune responses and consequently complicate common diagnostic approaches as well. However, to (cost)effectively eliminate these pathogens from populations at risk, accurate and timely diagnosis is essential.

As an example, paratuberculosis or Johne's disease (JD) in ruminants, caused by infection with Mycobacterium avium subspecies paratuberculosis (also called herein M. a. paratuberculosis, M. a. ssp paratuberculosis or M. avium ssp. paratuberculosis), leads to substantial economic losses, approximately 41 million euro annually, in the Netherlands. A serological survey on 378 dairy farms in the Netherlands revealed one or more ELISA-positive cows on 55% of the farms, the true prevalence at cow level being estimated between 2 and 7%.^{[1}] An as yet unknown number of goat and sheep flocks are infected as well, and potentially susceptible wildlife species may be involved in transmission.^{[2]} Furthermore, it has been suggested that M. a. paratuberculosis is involved in the etiology of Crohn's disease in humans.^{[3]} Thus, the presence of the bacteria in the environment and food products may have human health consequences. Both animal and human health aspects justify research into the immunopathogenesis of paratuberculosis aimed at improving diagnostic tools for control of the disease and eradication strategies.

In cattle, the course of the disease is as follows. Calves acquire the infection in the first months of life through oral uptake of colostrum, milk or feces of infected cows. They either successfully clear the infection or become subclinically infected for life. The subclinically infected animals shed the bacteria in their feces intermittently or continuously from an age of approximately two years onward. After an incubation period of four to five years, a proportion of the subclinically infected animals develops an incurable progressive form of protein-losing enteropathy with chronic diarrhea that is ultimately fatal.^{[4]}

Vaccination of calves in the first month of life prevents the development of the clinical stage of the disease and thus reduces economical damage. However, it does not result in elimination of mycobacteria since subclinically infected animals can still be detected in approximately the same frequency in vaccinated herds as compared to non-vaccinated herds. In addition, the current vaccination strategy interferes with bovine tuberculosis diagnostics. These serious drawbacks, both from an epidemiological and a human health point of view, currently limit, or even prohibit, the use of vaccination.^{[5]}

Accurate and timely diagnosis of the infection is a major problem and the lack thereof severely hampers attempts to control the disease at the farm level. The gold standard of diagnosis so far is culture of bacteria from feces of infected cattle older than two years. Apart from bacterial culture, specific immune responsiveness, as determined by in vitro IFN-γ tests^{[6]} or the so-called absorbed JD-ELISA,^{[7]} may identify infected animals two to three years after infection; however, diagnosis in animals younger than two years is still problematic mainly due to poor sensitivity of the current assays. None of the immunological assays are conclusive as a diagnostic tool for the whole period of the infection, as will be addressed below.^{[8]}

Many research projects aimed at improving diagnostic procedures and relatively few efforts have been taken to unravel the immuno-pathogenesis of this disease. Several studies indicated that the major pathological effects have an immunopathological background.^{[9]} Data, generally derived from diagnostic studies using only Purified Protein Derivative of M. a. paratuberculosis (PPD-P) as antigen, indicate that initial immune responses are primarily of the cell-mediated type (CMI). As the disease progresses, the humoral responses become increasingly apparent. The clinical stage of paratuberculosis is associated with a (complete) loss of protective, cell-mediated, responses. For these reasons, none of the immunological diagnostic procedures cover the whole period of infection; moreover, alternating positive and negative results for either of the assays have been obtained.^{[10]} This change from putative protective CMI to permissive humoral responses has recently been associated with a switch from the so-called Type 1 to Type 2 T-cell reactivity.^{[11, 12]} Type 1 responses, as described for murine model systems, are thought to be effective against intracellular pathogens, such as M. a. paratuberculosis, by IFN-γ production that activates macrophages, cytotoxic T-cells (CTL) and induces certain isotypes of antibodies. Expanding Type 2 T-cells give rise to mainly humoral responses involving different isotypes of antibodies and inhibit inflammatory responses as induced by type 1 T-cells. Antigen concentration, co-stimulatory molecules and micro-environmental factors, like various types of cytokines, regulate the balance between Type 1 and Type 2 responses.^{[13]} In cattle, as compared to inbred strains of mice, the dichotomy between Type 1 and Type 2 T-cells is less clear.^{[14]}

In bovine paratuberculosis, vaccinated animals and healthy shedders have abundant (peripheral) cell-mediated responses, but are not protected against infection.^{[15, 16]} At best, these responses correlate with preventing the progression of the disease to the clinical stage, indicating a much more complex balance between host and pathogen, than the Type 1 to Type 2 shift. Alternative to a shift may be apoptotic death of T-cells, rather than activation, as a consequence of interaction with infected macrophages.^{[17]} Destruction of reactive Type 1 T-cells by apoptosis may be a mechanism of in vivo switch from Type 1 to Type 2 reactivity and in this respect, it is relevant to study the nature of the interactions between the (M. a. paratuberculosis infected) macrophages and responding T-cells and to characterize immune responses to the different structural and secreted bacterial antigens processed and presented in different stages of the infection to elucidate the immunopathogenesis.

Despite many efforts in the field, no suitable test is available for (early) diagnosis of slow progressive infections such as mycobacterial infections. Alternating positive and negative results for several assays have been obtained in different stages of infection and, in early stages, often no diagnosis is possible at all.

### DISCLOSURE OF THE INVENTION

It is an object of the present invention to provide an improved method for detecting infection with a microorganism that causes slow progressive disease. It is particularly an object to provide a method for diagnosis of such infections during early, latent stages. Furthermore, it is an object to at least in part counteract such infections.

Although the invention is explained in more detail for detection of mycobacterial species, it is to be understood that a method of the invention is suitable for detection of any microbial species that causes slow progressive infectious disease in cellular organisms such as mammals.

The invention provides a method for detecting infection of an animal with a microorganism that causes a slow progressive disease, comprising providing the animal with a protein of the microorganism, or a functional part, derivative and/or analogue thereof, and measuring an immune response of the animal directed against the microorganism. Preferably, the microorganism comprises a member of a mycobacterial species.

It has been found by the present inventors that diagnosis of an infection with a microorganism that causes slow progressive disease, such as a mycobacterial species, is possible during early stages of infection if an animal is provided with a protein of the microorganism, or with a functional part, derivative and/or analogue thereof. The (mycobacterial) protein may be administered before, during and/or after infection. In one embodiment of the invention, a mycobacterial protein is administered before infection takes place. According to the invention, if administration of a mycobacterial protein is followed by a mycobacterial infection, strong booster immune response is evoked in the animal so that detection of the response is possible during early stages of the infection. Usually, a mycobacterial infection is undetectable for years, even after multiple exposures of an animal to Mycobacteria. Surprisingly, however, after administration of a mycobacterial protein, a mycobacterial subsequent infection is capable of eliciting an immune response that is strong enough to be measured, whereas multiple exposures to mycobacteria are not capable of inducing such strong booster reactions. As shown in the examples, exposure to mycobacteria can already be demonstrated within 14 days after administration of a mycobacterial protein. While repeated exposures to mycobacteria are not capable of eliciting a booster immune response that is strong enough to be detected in early stages, a mycobacterial infection after exposure to a mycobacterial protein is capable of eliciting a measurable immune response. Therefore, administration of a mycobacterial protein is a valuable tool for diagnosis of mycobacterial infections, even during an early, latent stage.

In another embodiment, a mycobacterial protein is administered during or after a mycobacterial infection has taken place. In that case, administration of the mycobacterial protein results in a measurable immune response. A detectable immune response is obtained after administration of the protein within short.

Hence, it is possible to test animals for mycobacterial infection with a method of the invention. If an animal is already infected, a measurable immune response is obtained after administration of a mycobacterial protein. If an animal appears to be uninfected (yet) with Mycobacteria, the animal can be routinely screened after certain time intervals. If the animal is infected in a later stage, a strong immune response will be elicited due to the formerly administered mycobacterial protein. The immune response can, for instance, be detected during such routine screening procedures.

An animal may be directly provided with a mycobacterial protein or functional part, derivative and/or analogue thereof by administration of the mycobacterial protein or functional part, derivative and/or analogue, preferably in the presence of a suitable adjuvant such as, for instance, Dimethyl dioctadecyl ammonium bromide (DDA), Specol or a double oil emulsion. In another embodiment, however, the animal is indirectly provided with the mycobacterial protein or functional part, derivative and/or analogue thereof, for instance, by administration of a nucleic acid encoding the mycobacterial protein or functional part, derivative and/or analogue. The nucleic acid, for instance, comprises DNA or RNA. Preferably, the nucleic acid comprises DNA. Upon expression of the nucleic acid by the animal's machinery, mycobacterial protein or functional part, derivative and/or analogue thereof will be present and a mycobacterial infection can be detected. The nucleic acid may be expressed in any cell type of the animal. In one embodiment, the expression only takes place in one or several specific tissue/cell types. The nucleic acid may be artificially adapted for expression in certain kind(s) of cells only.

By “a mycobacterial protein” is meant a proteinaceous molecule that is involved with a Mycobacterium. Such protein may, for instance, be a part of such Mycobacterium. Such protein may also be a protein that is produced and/or excreted by the Mycobacterium. A functional part of a Mycobacterial protein is defined as a part that has the same kind of properties in kind, not necessarily in amount. Preferably, the functional part has the same kind of immunogenic properties, though such immunogenic properties need not be equal in amount. By “immunogenic properties” is meant the capability to induce an immune response in a host. A functional part of a mycobacterial protein preferably comprises an epitope of the protein. More preferably, the functional part comprises a B-cell and/or T-cell epitope of the protein.

A “functional derivative of a protein” is defined as a protein that has been altered such that the immunogenic properties of the derivative are essentially the same in kind, not necessarily in amount. A derivative can be provided in many ways, for instance, through conservative amino acid substitution. As is known by a person skilled in the art, a substitution of one amino acid with another with generally similar properties (size, hydrophobicity, etc.) does not seriously affect the overall functioning of a proteinaceous molecule.

A person skilled in the art is well able to generate analogous compounds of a protein. This can, for instance, be done through screening of a peptide library. Such an analogue has essentially the same immunogenic properties of the protein in kind, not necessarily in amount.

As used in this application, the term “mycobacterial protein” is meant to comprise a functional part, derivative and/or analogue of the mycobacterial protein as well.

An animal may be a non-human animal such as cattle, or a human individual. Preferably, the animal comprises a mammal. In one preferred embodiment, the mammal comprises a human individual. In another preferred embodiment, the mammal comprises a ruminant.

An immune response of an animal directed against a Mycobacterium is defined herein as a reaction of the animal's immune system against the Mycobacterium. Preferably, the reaction is specifically directed against the Mycobacterium. The immune response preferably comprises T-cell and/or B-cell activation. In a preferred embodiment, a method of the invention is provided wherein the immune response directed against the Mycobacterium comprises an immune response specifically directed against the mycobacterial protein or functional part, derivative and/or analogue thereof.

In a preferred embodiment of the invention, the immune response is measured within twelve months after the animal has been infected by a mycobacterial microorganism. Early diagnosis facilitates the possibilities for early treatment and for early isolation of contaminated animals and/or contaminated material from such animals in order to prevent spreading of the disease.

An animal can be provided with a mycobacterial protein in various ways. The protein can, for instance, be administered with a nasal spray or orally. Alternatively, the protein can be administered via an injection, for instance, subcutaneous or intramuscular. Such injection preferably comprises a suitable adjuvant such as, for instance, Dimethyl dioctadecyl ammonium bromide (DDA), Specol or a double oil emulsion. The mycobacterial protein may furthermore be coupled to a suitable carrier, such as keyhole limpet hemocyanin (KLH) or an immunogenic conjugate of a protein such as ovalbumin. Other methods of providing an animal with a protein are known in the art, which may be used in a method of the invention. Preferably, the protein is administered by subcutaneous injection.

In the art, many protocols are known for measuring an immune response. In a preferred embodiment, (memory) B-cell activation is measured in a method of the invention. For instance, antibodies specifically directed towards mycobacteria can be detected with ELISA and/or a biosensor. In another preferred embodiment, memory T-cell activation is measured in a method of the invention. Activation of (memory) T-cells is preferably measured by using peripheral blood mononuclear cells (PBMCs) for evaluating lymphoproliferative responses with a ³H-thymidine incorporation assay. However, alternative methods are known in the art. Preferably, the antibodies and/or T-cells are specifically directed against the mycobacterial protein or functional part, derivative and/or analogue thereof.

In a preferred embodiment, a method of the invention is provided wherein the Mycobacterium comprises Mycobacterium avium. More preferably, the Mycobacterium comprises Mycobacterium avium subspecies paratuberculosis. In yet another preferred embodiment, a method of the invention is provided wherein the animal comprises a ruminant, especially cattle, sheep, goat, and/or deer. As is outlined above, mycobacterial infections in ruminants cause substantial economic losses. Early diagnosis in ruminants is therefore highly desired.

### BRIEF DESCRIPTION OF THE DRAWINGS

FIG. 1 depicts Hsp70-specific antibody responses of calves experimentally infected with M. a. paratuberculosis. The whole protein Hsp70 ELISA demonstrates that a difference exists between the serum antibody responses of the calves in group 2 (immunization, no infection), and group 4 (immunization and infection). None of the (un-)infected controls (groups 1 and 3) show a response to Hsp70 in this stage of the infection.

FIG. 2 illustrates Hsp70 peptide-specific antibody responses of calves experimentally infected with M. a. paratuberculosis. Antibody responses of calves, as measured by Luminex technology (response indicated as mean fluorescence index (MFI) on the Y axis), to selected synthetic peptides from M. a. paratuberculosis Hsp70. Panel A shows the response to a conserved part of the protein, which is present in many (myco)bacteria, demonstrating booster-effects by environmental bacterial species. Panels B and C show responses to peptides that are unique to M. a. paratuberculosis Hsp70. No response was observed to the peptide shown in B. In contrast, a clear response is shown in panel C, however, only by calves (G4) primed with M. a. paratuberculosis Hsp70 and subsequently exposed to the bacteria, thus demonstrating the diagnostic capabilities of the system.

FIG. 3 depicts recombinant protein production and recognition by monoclonal antibodies 7D9, 6G1B9, and 6G1F3 in western blot. Lane 1, protein size marker; Lane 2, purified recombinant Hsp70 protein; Lane 3, soluble protein fraction E. Coli Top 10 production strain; Lanes 4 through 9, intermediate purification samples; and Lane 10, protein size marker.

FIGS. 4A-4C show protein and peptide ELISA of the M. a. paratuberculosis Hsp70-recognizing monoclonal antibodies: epitope identification.

FIG. 4A shows peptide ELISA using, five amino acids overlapping, peptides of the M. a. paratuberculosis Hsp70 protein covering less-conserved parts of the protein. These peptides were N-terminally linked to a cysteine to enable coupling to covalink ELISA plates. Peptides were first tested in pools. Subsequently, positive pools were retested using the individual peptides to identify linear epitopes.

| | |
|---|---|
| Sequence P1: | cys-ITDAVITVPAYFND |
| Sequence P3: | cys-AQAGGPDGAAAGGG |
| Sequence P4: | cys-PDGAAAGGGSGSAD |

FIG. 4B depicts protein ELISA using 7D9. Testing homologues of M. a. paratuberculosis Hsp70 in whole protein ELISA using 7D9 results in the following cross-reactive responses.

FIG. 4C illustrates protein ELISA using 6G1B9. Testing homologues of M. a. paratuberculosis Hsp70, no known equivalent of the 6G1B9 target peptide in the sequence, BLAST of peptide sequence returns only M. a. paratuberculosis sequence.

FIG. 5 is NUC ID NO:1 (unique C-terminal sequence M. a. paratuberculosis Hsp70).

FIG. 6 is PEP ID NO:1 (sequence recognized by monoclonal 7D9).

FIG. 7 is PEP ID NO:2 (sequence recognized by monoclonals 6G1B9 and 6G1F3).

FIG. 8 is PEP ID NO:3 (sequence recognized by bovine B cells from M. a. paratuberculosis infected calves treated with a single Hsp70 immunization).

FIG. 9 shows flowcytometric detection of intact M a. paratuberculosis using the monoclonal antibodies 6G1B9.

FIG. 10 shows immunomagnetic isolation of M. a. paratuberculosis using magnetic beads coated with 6G1B9 antibodies, confirmed using genomic insertion sequence IS900-specific PCR.

FIG. 11 is an electronmicrograph showing surface binding of 6G1B9 using immunogold labeling.

FIG. 12 is a fecal culture score graph showing putative curative effect of M. a. paratuberculosis Hsp70 immunization. FIG. 12 indicates that the experimentally infected animals (groups 3 and 4) have a comparable cumulative fecal score at 126 days post infection. However, when comparing later time points, groups 3 and 4 significantly differ in fecal culture score. This is caused by the fact that in group 3, six animals intermittently shed bacteria contrary to a single shedding animal in group 4. The fecal culture score of animals in groups 1 and 2 (uninfected controls) represents the minimal score using this method. For comparison, a theoretical maximal score (max) is added indicating the cumulative score of a positive fecal culture at each time point.

FIG. 13 depicts M. a. paratuberculosis Hsp70 peptide p111-124 specific antibody responses of goat kids experimentally infected with M a. paratuberculosis. Indicated are M. a. paratuberculosis Hsp70 peptide p111-124 specific antibody responses of goat kids of group 2 (immunization, no infection) and group 4 (immunization, infection). No responses were observed in group 1 (no immunization, no infection) and group 3 (no immunization, infection) (not shown).

FIG. 14 are protein sequence comparisons between M. a. avium Hsp70 (TIGR) and M. a. paratuberculosis Hsp70 (Genbank AF254578).

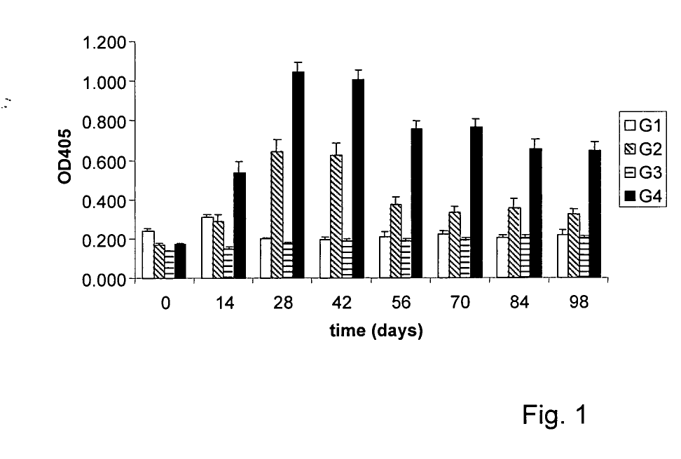

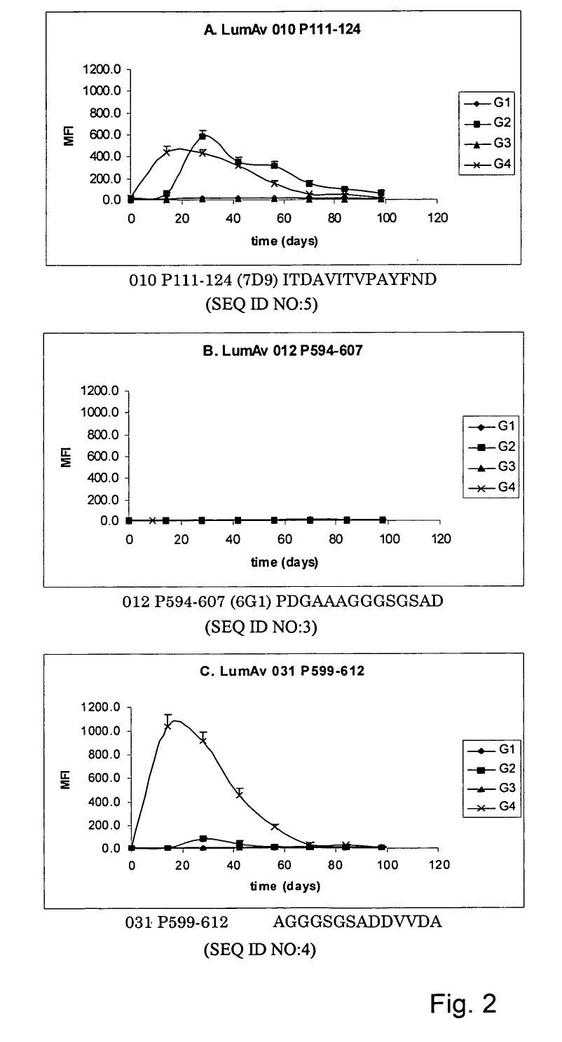

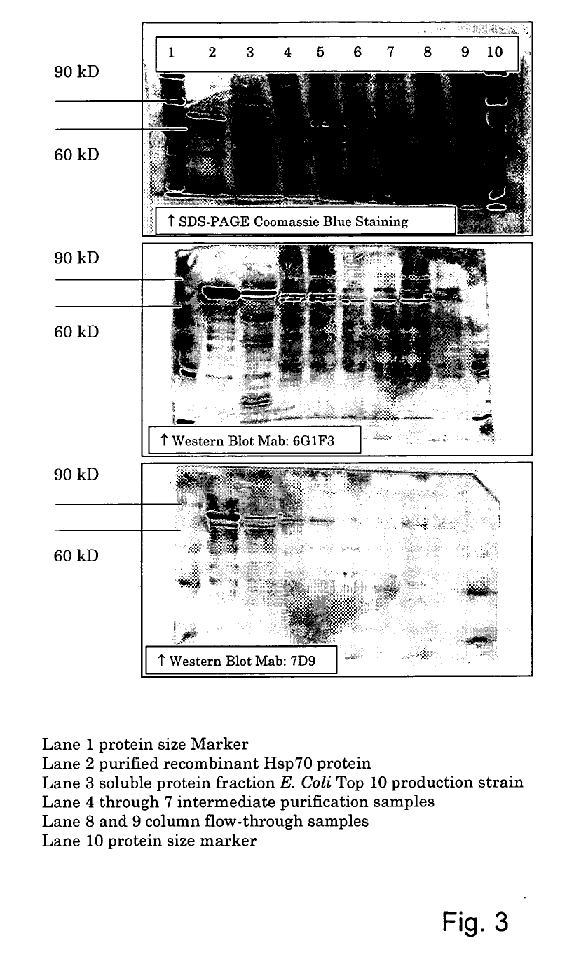

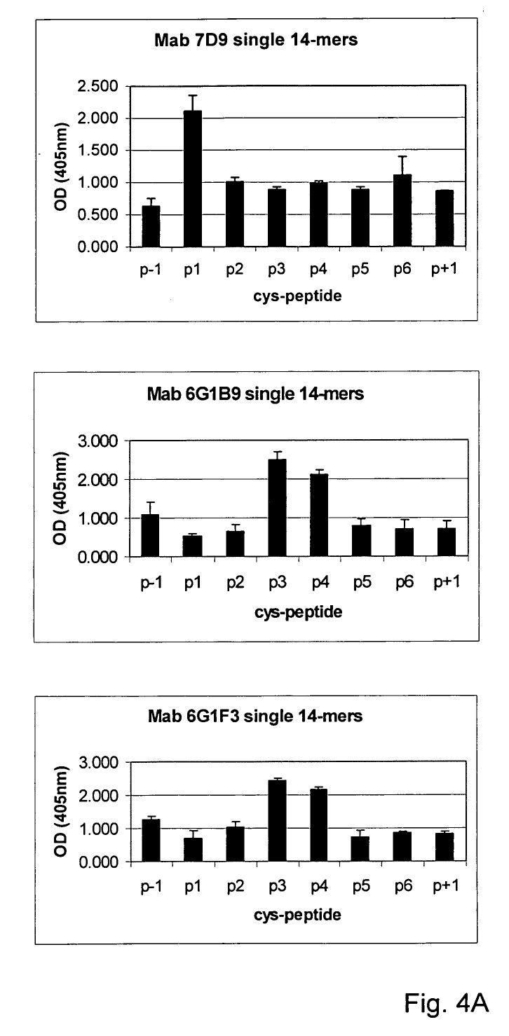

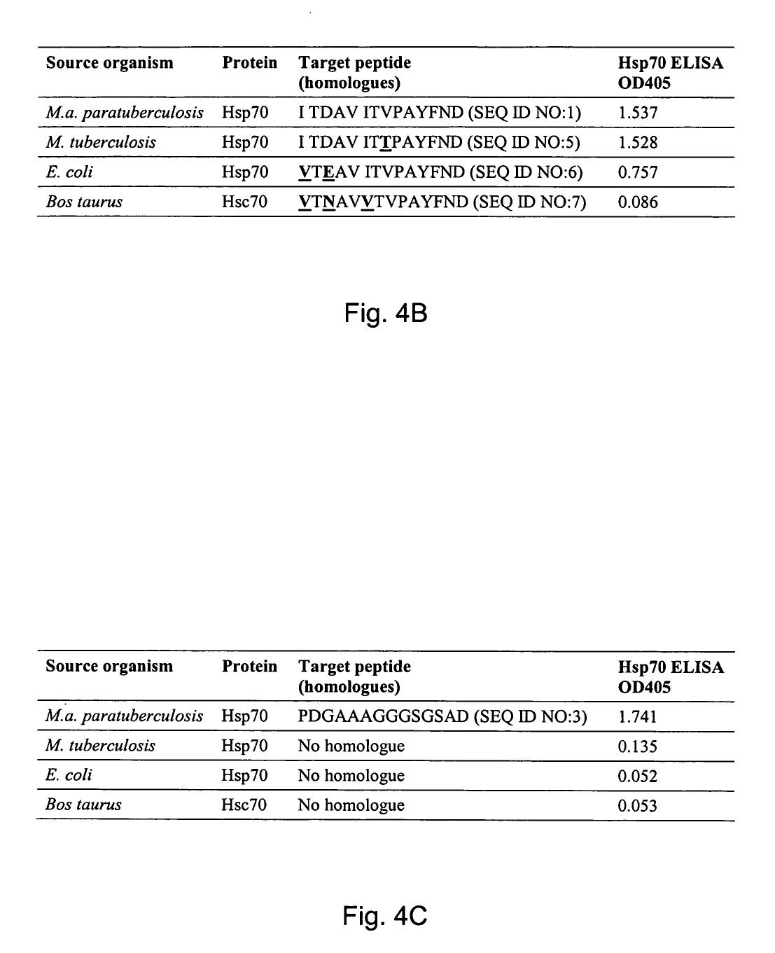

### DETAILED DESCRIPTION OF THE INVENTION

In one aspect, a method of the invention is provided wherein the mycobacterial protein or functional part, derivative and/or analogue thereof comprises mycobacterial heat shock protein 70 or functional part, derivative and/or analogue thereof. The functional part preferably comprises an epitope of the protein, such as a B-cell or T-cell epitope. More preferably, the functional part comprises a B-cell epitope. Preferred peptides comprising B-cell epitopes of the invention are depicted in FIGS. 6, 7 and 8.

Heat shock or stress proteins are cytoplasmatic proteins involved in intracellular processes of protein synthesis and translocation. The expression of Hsp is up-regulated during cellular stress; such as temperature, hypoxia, and tissue damage or inflammation. As such, they are essential in protection of both eukaryotic and prokaryotic cellular organisms against the potentially harmful effects of stress. Hsp have been defined as immunodominant, although most of them are highly conserved and ubiquitously distributed.^{[9]} Members of the 60, 70 and 90 kD Hsp families are involved in important aspects of bacterial infections^{[20]} and in autoimmune diseases.^{21]} Hsp act as immunological target structures, either by themselves because of an unusual expression pattern, or they are carrier proteins for immunogenic peptides.^{[22]} In addition to a classical major histocompatibility complex-(MHC-) restricted T-cell response, a major contribution in the recognition of heat shock proteins has been shown for non-MHC-restricted effector cells including gamma/delta TCR-positive T-lymphocytes and natural killer (NK) cells.^{[23]}

The Hsp65 and Hsp70 of M. a. paratuberculosis have been cloned and sequenced.^{[26, 27]} These evolutionary conserved proteins have not been studied intensively due to doubt as to their diagnostic value in conventional serological approaches.^{[28]} The present inventors, however, have extensively studied immune responses of cattle to Hsp65 and Hsp70 with regard to their role in the immunopathogenesis of bovine paratuberculosis. They have shown that both Hsps are immunodominant antigens in bovine paratuberculosis in terms of cell-mediated and antibody-mediated immune responses.

According to one embodiment, early, single immunization of young animals with recombinant 70 kD Heatshock protein (Hsp70) originating from Mycobacterium avium ssp. paratuberculosis (M. a. paratuberculosis), or with a functional part, derivative and/or analogue thereof, such as, for instance, a peptide comprising a B-cell epitope of Hsp70, strongly primes their immune system to subsequent exposure to mycobacterial organisms. This priming, when followed by exposure to a mycobacterial pathogen, induces immunological booster reactions that at least lead to activation of helper T-cells and B-cells and the production of antigen-specific immunoglobulins. This enables rapid and sensitive diagnosis of exposure to mycobacterial organisms using available technology for the measurement of (memory) T- and B-cell activation.

Alternatively, immunization with Hsp70 or a functional part, derivative and/or analogue thereof, after exposure to a mycobacterial organism, results in a strong immune response that can be measured. Now that a method of the invention is provided, other proteins or functional parts, derivatives and/or analogues thereof, optionally originating from different kinds of microorganisms causing a slow progressive disease, can be screened for their capability of eliciting a measurable immune response in a host after infection with the microorganism.

In one aspect, a method of the invention is provided wherein the protein or functional part, derivative and/or analogue thereof comprises a surface-associated protein or functional part, derivative and/or analogue thereof. After exposure to the microorganism, the host's immune system is immediately exposed to such surface-associated protein. In a preferred embodiment, the protein comprises a surface-associated mycobacterial protein. According to the present invention, Mycobacterium avium subspecies paratuberculosis heat shock protein 70 is a surface-associated mycobacterial protein and, hence, very suitable for a method of the invention. Likewise, Mycobacterium avium Hsp 60 is associated with the surface of M. avium.

In yet another preferred embodiment, the protein is recombinantly produced. This allows for controlled production of the protein. Moreover, it is possible to influence one or more properties of such protein. For instance, the protein may be rendered more immunogenic. This can, for instance, be done by generating a fusion protein comprising the protein and another immunogenic moiety, such as a T-helper cell epitope.

In one aspect, the invention provides an isolated peptide comprising a B-cell epitope of a Mycobacterium avium subspecies paratuberculosis heat shock protein 70 or a functional part, derivative and/or analogue thereof. A peptide comprising a B-cell epitope is particularly suitable for use in a method of the invention. It is not necessary to administer a whole mycobacterial protein to an animal because a functional part of such protein, such as a peptide comprising a B-cell epitope, is also capable of inducing a strong immune response after a mycobacterial infection. A peptide of the invention preferably varies in length from about five to about 500 amino acid residues. More preferably, the peptide has a length of about five to about 100 amino acid residues, most preferably about five to about 20 amino acid residues. Administration of a peptide of the invention instead of a whole mycobacterial protein is preferred, because undesirable side effects of a whole mycobacterial protein can be at least partly diminished by a peptide of the invention. For instance, a part of the mycobacterial protein responsible for an adverse side effect can be omitted in a peptide of the invention.

Moreover, a peptide of the invention is suitable for detecting Mycobacterium-directed antibodies. A mycobacterial infection of an animal can be detected by determining whether Mycobacterium-directed antibodies are present in a sample of the animal. Such antibodies can, for instance, be detected by common immunofluorescence, immunoblot, ELISA and/or biosensor techniques, using a peptide of the invention. As is known in the art, many alternative protocols exist for determining whether a peptide of the invention is bound to Mycobacterium-directed antibodies. For instance, affinity chromatography can be used.

In one aspect, a peptide of the invention is provided, wherein the epitope comprises at least a functional part of a sequence as depicted in FIG. 6. The sequence as depicted in FIG. 6 was identified by the present inventors and appears to be a linear epitope in Hsp70. This sequence is present in M. avium and in M. avium ssp paratuberculosis. In another aspect, a peptide of the invention is provided wherein the epitope comprises at least a functional part of a sequence as depicted in FIG. 7. The sequence as depicted in FIG. 7 is a M. avium ssp paratuberculosis epitope. In yet another aspect, a peptide of the invention is provided wherein the epitope comprises at least a functional part of a sequence as depicted in FIG. 8, which is a M. avium ssp paratuberculosis epitope as well.

A peptide comprising a sequence as depicted in FIGS. 6, 7 and/or 8 is especially suitable for a method of diagnosis of the invention. Mycobacterium-directed antibodies can be detected. It has, therefore, not only become possible to detect a mycobacterial infection in an early stage, but also to distinguish between mycobacterial species. For instance, a peptide comprising a sequence as depicted in FIGS. 7 and/or 8 is capable of specifically demonstrating M. avium ssp paratuberculosis-directed antibodies, whereas a peptide comprising a sequence as depicted in FIG. 6 is capable of demonstrating a M. avium infection.

A peptide comprising a sequence as depicted in FIGS. 6, 7 and/or 8 is also suitable for eliciting antibodies against M. avium. This can be performed for vaccination purposes, for instance, in human beings and in cattle.

A peptide comprising a sequence as depicted in FIGS. 7 and/or 8 is capable of eliciting an immune response specifically directed against M. avium ssp paratuberculosis, which does not cross-react against other M. avium species. The peptide is, for instance, capable of eliciting antibodies that do not cross-react against antigens of other M. avium species. Therefore, a peptide comprising a sequence as depicted in FIGS. 7 and/or 8, or a functional part, derivative or analogue of the peptide, is particularly suitable for use in a marker vaccine. A marker vaccine allows discrimination between vaccinated animals and animals that are naturally infected. A marker vaccine comprising a peptide comprising a sequence as depicted in FIGS. 7 and/or 8, or a functional part, derivative or analogue thereof, allows discrimination between vaccinated animals and animals infected by a M. avium species other than M. avium ssp paratuberculosis. The marker vaccine preferably also comprises an additional immunogenic component, such as a T-cell epitope.

Contrary to conventional vaccines, the marker vaccine does not raise antibodies against M. avium species other than M. avium ssp paratuberculosis. Hence, when animals are screened for the presence of an immune response against M. avium species other than M. avium ssp paratuberculosis, vaccinated animals with a marker vaccine of the invention do not give false-positive test results. Contrary, animals vaccinated with conventional vaccines often give false-positive test results because of cross-immunity. The invention thus provides a vaccine comprising a peptide comprising a sequence as depicted in FIGS. 7 and/or 8, or a functional part, derivative or analogue thereof. The invention also provides a vaccine comprising a nucleic acid sequence encoding a peptide comprising a sequence as depicted in FIGS. 7 and/or 8, or a functional part, derivative or analogue thereof. A vaccine of the invention preferably also comprises an additional immunogenic component, such as a T-cell epitope. A use of a peptide comprising a sequence as depicted in FIGS. 7 and/or 8, or a functional part, derivative or analogue thereof, for the preparation of a vaccine is also herewith provided, as well as a use of a nucleic acid sequence encoding a peptide comprising a sequence as depicted in FIGS. 7 and/or 8, or a functional part, derivative or analogue thereof, for the preparation of a vaccine. In one embodiment, a vaccine of the invention is used for discriminating between vaccinated animals and animals that are infected by M. avium ssp paratuberculosis. This is, for instance, done by using an epitope in a vaccine of the invention which is not naturally exposed to an animal's immune system during M. avium ssp paratuberculosis infection.

In one embodiment, a non-human animal is provided with a peptide of the invention in order to generate and collect antibodies directed against M. avium. The antibodies are preferably directed against M. avium ssp paratuberculosis. Obtained antibodies are used for diagnosis (demonstrating the presence of mycobacterial antigens in a sample), passive immunization, etc.

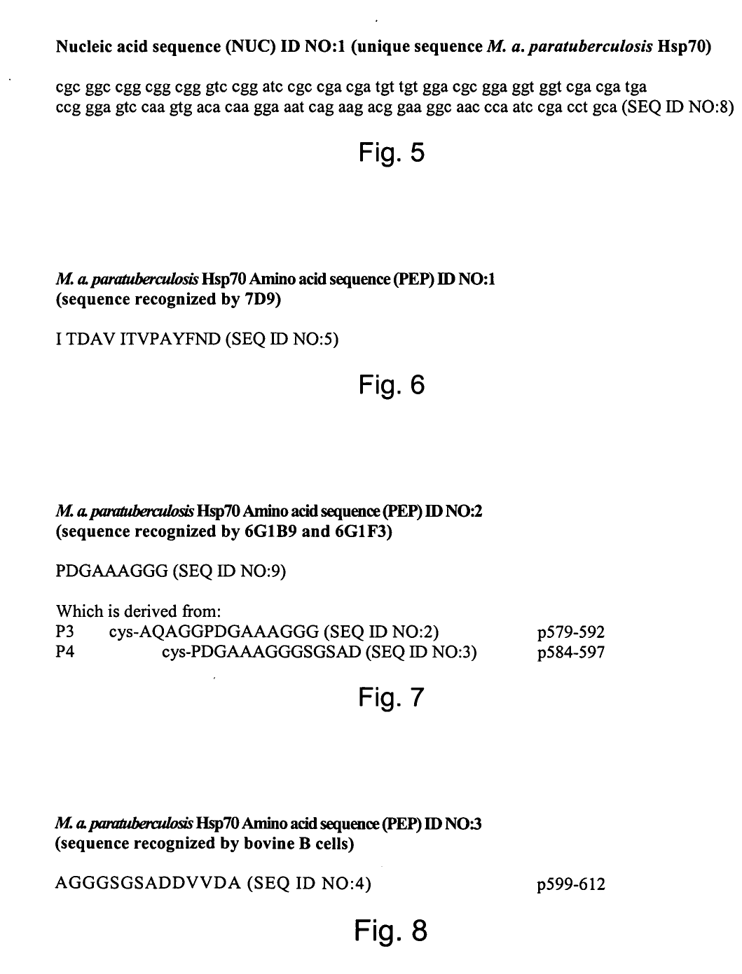

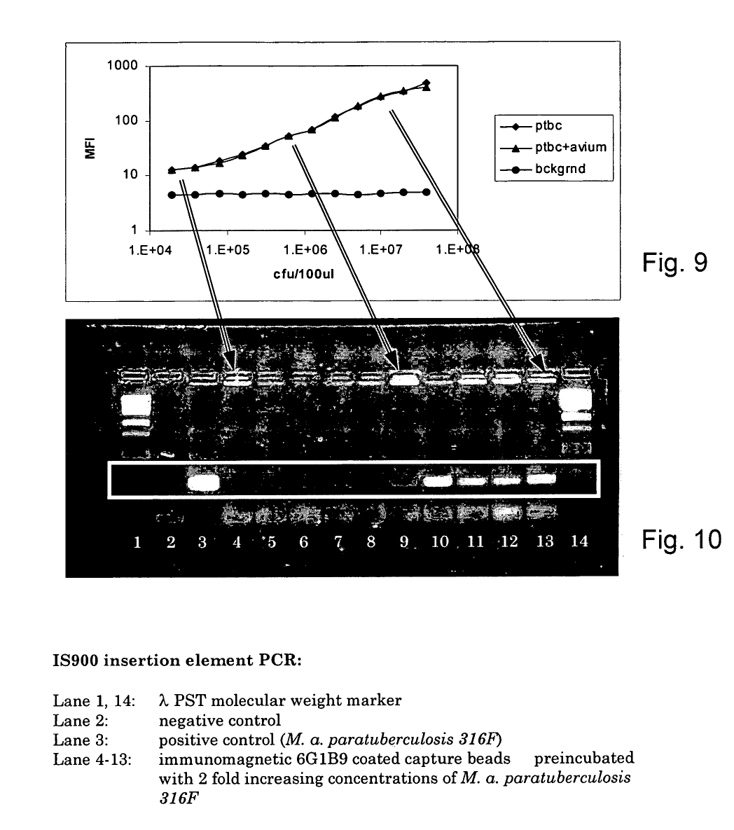

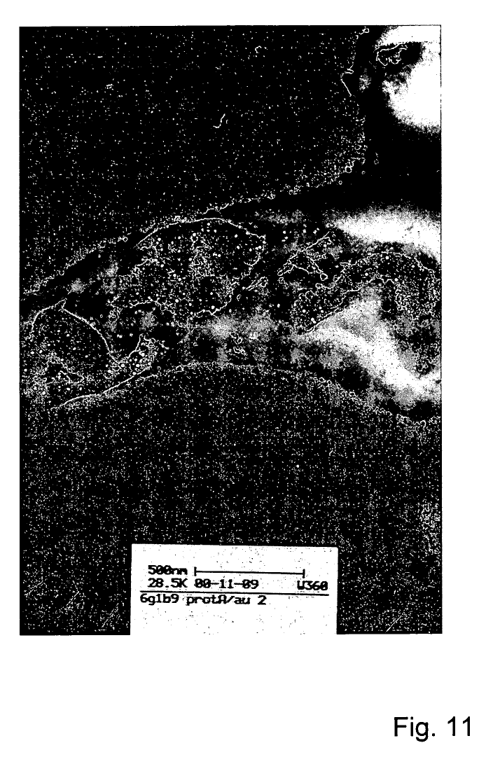

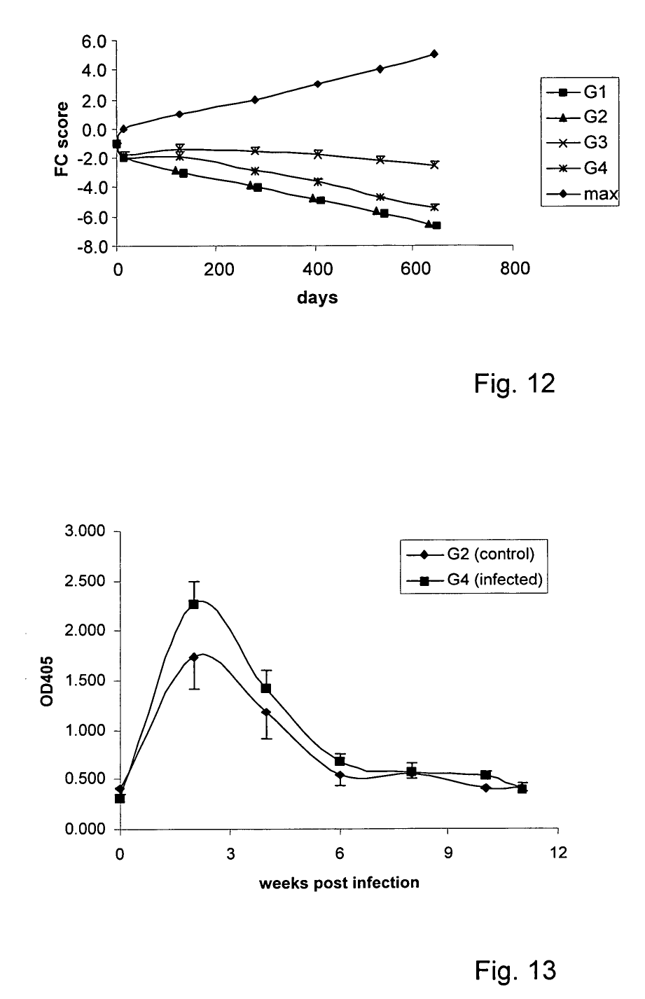

Also provided is an isolated nucleic acid molecule encoding a peptide of the invention, or a functional part, derivative and/or analogue thereof. Preferably, the nucleic acid encodes a peptide varying in length from about five to about 500 amino acid residues. More preferably, the nucleic acid encodes a peptide varying in length from about five to about 100 amino acid residues, most preferably from about five to about 20 amino acid residues. Such isolated nucleic acid is suitable for generation of a peptide of the invention. In yet another aspect, an isolated nucleic acid molecule comprising at least a functional part of a sequence as depicted in FIG. 5 is provided. A nucleic acid sequence as depicted in FIG. 5 is particularly indicative for M. avium ssp paratuberculosis. No other mycobacterial species/subtypes are known to comprise a nucleic acid sequence as depicted in FIG. 5. Hence, with a nucleic acid molecule comprising the sequence, it has become possible to distinguish M. avium ssp paratuberculosis from other mycobacterial species. It has, therefore, not only become possible to detect a mycobacterial infection in an early stage, but also to distinguish between mycobacterial species. For instance, a sample from a potentially infected animal can be screened for the presence of a nucleic acid molecule capable of hybridizing with a nucleic acid molecule of the invention. This can, for instance, be done by common Northern/Southern blot procedures, using a nucleic acid molecule of the invention as a probe. Alternatively, a PCR can be performed using (part of) a nucleic acid of the invention as a primer. Many other methods for detecting a specific nucleic acid sequence within a sample are known in the art, which need no further explication here. A nucleic acid of the invention may comprise DNA, RNA and/or an analogue thereof. In one embodiment, a nucleic acid of the invention comprises peptide nucleic acid (PNA). PNA is an analogue of DNA in which the phosphodiester backbone has been replaced by a pseudo-peptide chain. PNA mimics the behavior of DNA and binds complementary nucleic acid strands. The neutral backbone of PNA often results in stronger binding and greater specificity than normally achieved. In addition, the chemical, physical and biological properties of PNA can be exploited to produce, for instance, powerful molecular probes, biosensors and antisense agents.

A peptide of the invention can be administered to an animal in order to induce a measurable immune response after a mycobacterial infection. Peptides comprising an immunogenic part of a mycobacterial protein such as a B-cell epitope can be generated, for instance, by conventional peptide synthesis techniques or with recombinant expression methods. If such peptides are administered to an animal instead of the whole mycobacterial protein, side effects can be at least partly reduced. Such peptide may, for instance, be less cross-reactive. Hence, in one aspect, a method of the invention is provided wherein the animal is provided with a peptide or a functional part, derivative and/or analogue thereof of the invention. In one embodiment, the animal is provided with a peptide comprising at least part of a sequence as depicted in FIGS. 7 and/or 8. Since a sequence as depicted in FIGS. 7 and/or 8 is particularly indicative for M. avium ssp paratuberculosis, diagnosis of this species is provided in this embodiment.

An animal can also be provided with a nucleic acid of the invention, for instance, by gene therapy. A gene delivery vehicle can be used for this purpose, although many more methods are known in the art. A nucleic acid of the invention is preferably provided with a suitable promoter that enables expression of the nucleic acid sequence within the animal, more preferably, in specific kind(s) of cells of the animal. After incorporation and expression of the nucleic acid, generated expression product is capable of inducing an enhanced immune response after a mycobacterial infection. Such animals can then be regularly screened. A mycobacterial infection can be detected in an early stage. Preferably, the nucleic acid sequence encodes a peptide of the invention comprising an immunogenic part of a mycobacterial protein, more preferably comprising a B-cell epitope of a mycobacterial protein. The mycobacterial protein preferably comprises a heat shock protein. Most preferably, the nucleic acid sequence encodes a peptide of the invention comprising a B-cell epitope of M. avium ssp paratuberculosis heat shock protein 70. Hsp70 epitopes are depicted in FIGS. 6, 7 and 8. In one aspect, the nucleic acid comprises at least a functional part of a sequence as depicted in FIG. 5.

A gene delivery vehicle comprising a nucleic acid of the invention is also herewith provided, as well as a use of a gene delivery vehicle comprising a nucleic acid encoding a mycobacterial protein, or a functional part, derivative and/or analogue thereof, for detecting infection of a Mycobacterium in an animal. Preferably, the Mycobacterium comprises Mycobacterium avium, more preferably, Mycobacterium avium subspecies paratuberculosis. A gene delivery vehicle comprising a nucleic acid of the invention can be generated using known methods in the art.

By “at least a functional part of a nucleic acid of the invention” is meant a part of the nucleic acid, at least 20 base pairs long, preferably at least 50 base pairs long, more preferably at least 100 base pairs long, comprising at least one characteristic (in kind, not necessarily in amount) as a nucleic acid of the invention. The characteristic preferably comprises an expression characteristic. Preferably, a functional part of a nucleic acid sequence as depicted in FIG. 5 is still capable of distinguishing M. avium ssp paratuberculosis from other species. In one embodiment, nucleic acid from M. avium ssp paratuberculosis is capable of annealing to the functional part (preferably under stringent conditions), whereas nucleic acid from other Mycobacterium species is not. Moreover, an expression product of such functional part is preferably particularly indicative for M. avium ssp paratuberculosis.

By “at least a functional part of an epitope” is meant an amino acid sequence that is capable of eliciting the same immune response in kind, not necessarily in amount, as the epitope.

In one aspect, the invention provides a diagnostic kit comprising:
a protein of a microorganism that causes a slow progressive disease, or a functional part, derivative and/or analogue of the protein, and
optionally, means for measuring an immune response of an animal.

The invention also provides a diagnostic kit comprising:
a mycobacterial protein, or a functional part, derivative and/or analogue thereof, and
optionally, means for measuring an immune response of an animal.

In another embodiment, the invention provides a diagnostic kit comprising:
a nucleic acid comprising a sequence encoding a protein of a microorganism that causes a slow progressive disease or a functional part, derivative and/or analogue of the protein, and
optionally, means for measuring an immune response of an animal.
   Preferably, the microorganism comprises a Mycobacterium. More preferably, the Mycobacterium comprises Mycobacterium avium. Most preferably, the mycobacterial protein comprises a Mycobacterium avium subspecies paratuberculosis protein. In one embodiment, a diagnostic kit of the invention is provided, wherein the mycobacterial protein comprises Mycobacterium avium subspecies paratuberculosis heat shock protein 70. In yet another aspect, a diagnostic kit is provided comprising a peptide or a nucleic acid of the invention.

A diagnostic kit of the invention is suitable for early detection of an infection with a microorganism that causes a slow progressive disease. In one embodiment, the infection comprises a mycobacterial infection. For instance, such diagnostic kit can be used by farmers to detect Mycobacterium avium ssp paratuberculosis in cattle. Alternatively, a diagnostic kit of the invention can be used to detect mycobacterial infection in humans, such as infection with M. tuberculosis and M. leprae. A mycobacterial protein, a functional part, derivative and/or analogue thereof capable of inducing a measurable immune response after a mycobacterial infection, and/or a peptide of the invention can be administered to a human individual or to a non-human animal. Subsequently, an immune response can be detected if the human or animal is infected with mycobacteria. It is, of course, preferred to use a protein or peptide derived from the same Mycobacterium species that is tested for. However, since cross-reactions are possible, for instance, with several M. avium heat shock proteins, this is not always necessary. After a mycobacterial infection is detected, it is often desirable to identify the kind of mycobacterial species. This can, for instance, be done with a nucleic acid molecule comprising a sequence as depicted in FIG. 5. As is described above, this sequence is indicative for M. avium ssp paratuberculosis. The nucleic acid molecule can, therefore, be used to determine whether nucleic acid from an unknown mycobacterial species is capable of annealing to the nucleic acid molecule under stringent conditions. If nucleic acid present in a sample is capable of annealing under stringent conditions, it is indicative for the presence of M. avium ssp paratuberculosis nucleic acid in the sample. A peptide comprising a sequence as depicted in FIGS. 7 and/or 8 is capable of specifically demonstrating the presence of M. avium ssp paratuberculosis as well.

In order to detect mycobacterial nucleic acid in a sample, the nucleic acid often has to be amplified. Amplification can be performed with common methods in the art, such as, for instance, PCR, Nasba, SDA and/or TMA, which are well known in the art and need no further explanation here. An amplification reaction can be performed with non-specific primers, after which the presence of a nucleic acid of interest can be detected with a specific probe. The amplification reaction can alternatively be performed with specific primers, or with a combination of specific and non-specific primers. In order to detect mycobacterial nucleic acid, more preferably, Mycobacterium avium ssp paratuberculosis nucleic acid, it is preferred to use at least part of a nucleic acid of the invention as a primer or probe. It is clear that, if a primer or probe is meant to anneal to a sense strand of mycobacterial nucleic acid, the primer or probe preferably comprises the anti-sense strand sequence of the mycobacterial nucleic acid sequence, and vice versa. In one aspect, the invention provides a primer or probe capable of hybridizing to a nucleic acid molecule of the invention.

An isolated antibody, or a functional part, derivative and/or analogue thereof, capable of specifically binding a peptide of the invention, is also herewith provided. A functional part of an antibody is defined as a part that has essentially the same properties of the antibody in kind, not necessarily in amount. The functional part is preferably capable of binding at least one same antigen, as compared to the antibody. However, the functional part may bind such antigen to a different extent. A functional part of an antibody, for instance, comprises a FAB fragment or a single chain antibody. A derivative of an antibody is defined as an antibody that has been altered such that the immunogenic properties of the antibody are essentially the same in kind, not necessarily in amount. A derivative can, for instance, be provided through conservative amino acid substitution. A derivative of the invention also comprises a fusion protein with essentially the same immunogenic properties of the antibody in kind, not necessarily in amount. An analogue of an antibody can, for instance, be found through screening of a peptide library. Such an analogue has essentially the same immunogenic properties of the antibody in kind, not necessarily in amount.

An antibody or functional part, derivative and/or analogue of the invention can be used to detect the presence of a microorganism that causes a slow progressive disease, such as, for instance, a Mycobacterium, in a sample. The antibody or functional part, derivative and/or analogue can, for instance, be coated on an ELISA microtiter plate or a biosensor, after which the plate or biosensor can be incubated with a sample from a human individual or a non-human animal such as a ruminant. If the antibody appears to have bound to some component of the sample, it is indicative for the presence of proteins, or fragments thereof, of the microorganism in the sample and, hence, for infection with the microorganism. The antibody may be obtained from an infected individual. The antibody can, for instance, be isolated from a sample obtained from the infected individual. Alternatively, a non-human animal can be provided with the microorganism in order to induce an immune response. Produced antibodies can subsequently be collected from the animal. Alternatively, the antibody can be recombinantly produced, for instance, by microorganisms, cell lines or transgenic animals. The antibody may as well be synthesized using common techniques such as solid phase synthesis. As used herein, the term “antibody” is also meant to comprise a functional part, derivative and/or analogue of the antibody.

If a Mycobacterium-directed antibody is isolated from a sample obtained from an individual, it is in itself indicative of mycobacterial infection of the individual.

A measurable immune reaction obtained with a method of the invention is not only capable of demonstrating infection, it is also capable of, at least in part, counteracting the infection. For instance, a mycobacterial infection can be, at least in part, treated by inducing an enhanced immune response in an animal with a method of the invention. In one aspect, the invention, therefore, provides a method for, at least in part, treating an infection of a Mycobacterium in an animal, comprising providing the animal with a mycobacterial protein, or functional part, derivative and/or analogue thereof, and/or with a peptide of the invention. Preferably, the Mycobacterium comprises Mycobacterium avium, more preferably, Mycobacterium avium subspecies paratuberculosis.

The Mycobacterial protein preferably comprises heat shock protein 70.

A pharmaceutical composition comprising a protein or functional part, derivative and/or analogue of the invention is also herewith provided. Preferably, the protein or functional part, derivative and/or analogue comprises a mycobacterial protein or functional part, derivative and/or analogue. The pharmaceutical composition preferably comprises a suitable adjuvant. In one embodiment, the pharmaceutical composition comprises a peptide of the present invention. The peptide preferably comprises a sequence as depicted in FIGS. 7 and/or 8.

In another aspect, a pharmaceutical composition comprising a nucleic acid of the invention is provided. Preferably, the nucleic acid encodes a mycobacterial protein, or functional part, derivative and/or analogue thereof. The pharmaceutical composition can, for instance, be used for gene therapy, as described above.

Preferably, a pharmaceutical composition of the invention is provided, wherein the Mycobacterium comprises Mycobacterium avium. More preferably, the Mycobacterium comprises Mycobacterium avium subspecies paratuberculosis. In one aspect of the invention, the Mycobacterial protein comprises a Mycobacterium avium subspecies paratuberculosis heat shock protein 70 or functional part, derivative and/or analogue thereof. A method for at least in part treating an infection of an animal with a microorganism that causes a slow progressive disease comprising providing the animal with a pharmaceutical composition of the invention is also herewith provided. The microorganism preferably comprises a Mycobacterium, more preferably, M. avium, most preferably, M. avium ssp paratuberculosis.

The following examples are meant to illustrate the present invention. They do not limit the scope of the invention in any way.

### EXAMPLES

Material and Methods

Experimental Paratuberculosis Infections in Calves

Animals and Experimental Design

A total of 40 calves (aged 29±9 days at the start of the experiment) were randomly assigned to one of the following four experimental groups.

| Group | n | Infection | Immunization |
|---|---|---|---|
| G1 | 10 | no | no |
| G2 | 10 | no | yes |
| G3 | 10 | yes | no |
| G4 | 10 | yes | yes |

The calves were raised using conventional procedures and feeds, and were checked daily for general health. Calves in groups 1 and 2 were physically separated from calves in groups 3 and 4, and rigorous hygienic measures were taken to prevent infection of the control groups. Body weight was recorded every two weeks. Blood samples were taken every two weeks. Fecal samples were taken seven times during the experiment, at days 0, 14, 126, 280, 406, 532 and 644. The shedding of M a. paratuberculosis in feces was scored using the following equation: FCscore(t)=C(t)+FCscore(t-1). The fecal culture score (FCscore) is a cumulative score where the score at time point (t) equals the culture result at time point (t) plus the score from the previous date, in which: C(t)=−1 (negative culture result), 0 (no result), or +1 (positive culture result).
Infection of Calves

Calves assigned to groups 3 and 4 were infected orally using feces from an M. a. paratuberculosis infected cow which was characterized as a shedder by fecal culture of the mycobactin-J-dependant and IS900 PCR-positive organism. The calves received nine doses of 20 grams of feces during the first 21 days of the experiment at regular intervals.

Experimental Paratuberculosis Infections in Goats

Animals and Experimental Design

A total of 30 goat kids (aged 9±3 days at the start of the experiment) were randomly assigned to one of the following four experimental groups.

| Group | n | Infection | Immunization |
|---|---|---|---|
| G1 | 7 | no | no |
| G2 | 8 | no | yes |
| G3 | 7 | yes | no |
| G4 | 8 | yes | yes |

The goat kids were raised using conventional procedures and feeds, and were checked daily for general health. Goat kids in groups 1 and 2 were physically separated from goat kids in groups 3 and 4, and rigorous hygienic measures were taken to prevent infection of the control groups. Blood samples were taken weekly. Tissue samples (distal ileum and ileocecal lymphnode) were collected at the end of the experiment (week 12) for bacteriological culture.
Infection of Goat Kids

Goat kids assigned to groups 3 and 4 were infected orally using cultured M. a. paratuberculosis G195 strain bacteria, originally isolated from a M. a. paratuberculosis-infected goat with clinical signs of infection. The strain has been characterized as a mycobactin-J-dependant and IS900 PCR-positive organism. The goat kids received five doses of approximately 1×10⁹ cfu each, during the first 15 days of the experiment at regular intervals.

Immunization of Calves and Goat Kids

Calves and goat kids assigned to groups 2 and 4 were immunized once at the start of the experiment (day 0). The immunization consisted of the administration of 200 μg (calves) or 100 μg (goat kids) of recombinant M. a. paratuberculosis Hsp70 in dimethyl dioctadecyl ammonium bromide (DDA) adjuvant (Sigma Aldrich, USA), subcutaneously in the dewlap. Recombinant M. a. paratuberculosis Hsp70 was produced as published previously.^{[29]}

Synthetic Peptides

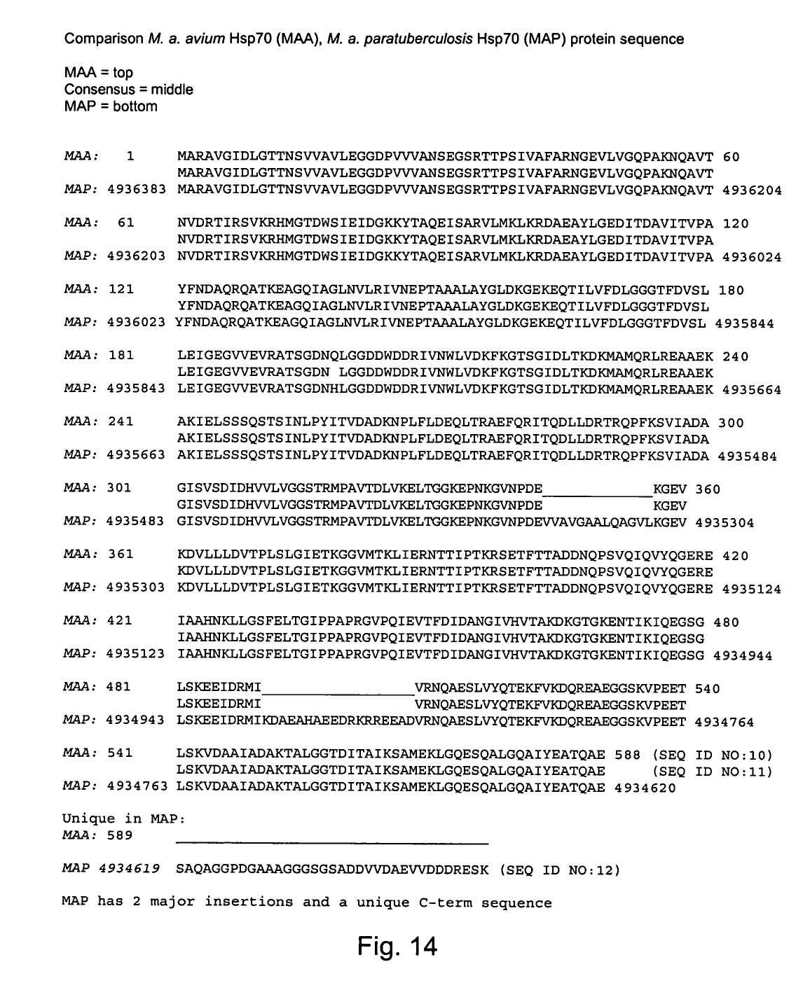

Based on sequence comparisons between M. a. avium Hsp70 (TIGR) and M. a. paratuberculosis Hsp70 (Genbank AF254578), illustrated in FIG. 14, synthetic overlapping 14-mer peptides were synthesized from the regions that are unique to M. a. paratuberculosis Hsp70 using simultaneous multiple peptide synthesis (SMPS). One peptide from a conserved region was synthesized also. These peptides were all biotinylated using N-terminal biotinylation during SMPS.

Serology of Calves and Goat Kids

Serological responses to recombinant M. a. paratuberculosis Hsp70 protein were measured using a previously described ELISA technique.^{[29]} Peptide specific serological responses in calves were monitored using fluorescently labeled microspheres coated with avidin (LumAv beads, Luminex, USA). In total, 20 uniquely labeled microspheres were incubated with one M. a. paratuberculosis Hsp70 peptide each. The beads were subsequently mixed and incubated with serum samples. Protein-A conjugated to R-PE (Prozyme, USA) was used as a reporter to detect antibodies recognizing the peptides coupled to the beads. The antibody response was analyzed in a multiplex detection system according to instructions from the manufacturer (Luminex L100, xMAP, Luminex, USA).

Peptide-specific serological responses in goat kids were monitored using streptavidin-coated microtiter plates (Pierce, USA) to which the biotinylated peptides were coupled according to instructions provided by the manufacturer. Subsequently, plates were blocked using a blocking reagent for ELISA (Roche, Germany), washed and 1:10 prediluted serum samples were added in duplicate. Plates were incubated for 30 minutes and subsequently washed. Next, a peroxidase-conjugated anti-goat antibody (Sigma Aldrich, USA) was added to the wells and incubated for 30 minutes. Finally, plates were washed, color was developed using ABTS substrate solution (Roche, Germany), and optical density was measured at 405 nm wavelength using a spectrophotometric ELISA reader (Biorad, USA).

Generation of Monoclonal Antibodies

Monoclonal antibodies to recombinant M. a. paratuberculosis Hsp70 were generated as follows. Balb/c mice were immunized with 100 μg of recombinant protein in incomplete Freunds adjuvant (IFA) three times subcutaneously with three-week intervals. A final booster immunization was given by intravenous administration of 100 μg Hsp70 three days prior to sacrificing the mice. A single cell suspension was prepared from the spleen and the splenocytes were fused to the HAT-sensitive SP2\0 myeloma fusion partner. Hybridomas were generated using conventional limiting dilution techniques. Monoclonal antibodies were purified using a thiophillic agarose affinity column (AFFI-T, Kem-En-Tec) and isotyped using the Mouse Hybridoma Subtyping Kit according to the producer's manual (Roche, Germany) and screened for antigen specificity using recombinant M. a. paratuberculosis Hsp70 protein and synthetic peptides.

Bacteria and Protein ELISA for Monoclonal Antibody Characterization

Protein antigens used were M. a. paratuberculosis Hsp70 (produced as described above) and M. tuberculosis Hsp70, E. coli Hsp70 and Bos Taurus Hsc70 (all from Stressgen, Canada). Next, 96-well plates (Corning Costar, USA) were coated with 1 μg of antigen or 100 μl of washed bacteria, diluted in sodium bicarbonate buffer for 30 (antigens) or 60 (bacteria) minutes at room temperature, while shaking at 300 rpm on an electronic IKA MTS shaker (IKA, USA). All subsequent incubations were performed for 30 minutes shaking at room temperature. After each incubation step, plates were washed three times with PBS containing 0.01% Tween 20. Wells were blocked with 200 μl of Post Coating Buffer (PCB) (Roche, Germany). Murine sera or pure antibody solutions were (serially) diluted in PCB. This was followed by incubation with peroxidase-conjugated Goat anti-Mouse antibody (Roche, Germany) 1:2000 diluted in PCB. Finally, 100 μl ABTS substrate buffer (Roche, Germany) was added to each well. The OD 405 nm was measured after ten minutes using an automated ELISA reader (Biorad, USA).

Synthetic Peptide ELISA for Monoclonal Antibody Characterization

A 96-well CovaLink NH F8 plate (Nunc, USA) was coated with 100 μl of 0.5 mM SPDP in 2-propanol, diluted in PBS for 30 minutes at 37° C. After incubation, the plate was washed two times with redistilled water. All reagents were used in a volume of 100 μl/well and all subsequent incubations were performed at 37° C. The different cysteine-linked peptides were diluted in 0.1 M Tris-HCl, pH 8.0 at a concentration of 15 μg/ml, directly before transfer to wells. Peptides were incubated for 60 minutes. After this and all subsequent incubations, the plates were washed three times with tap water. Wells were blocked with 200 μl of PCB (Roche, Germany) for 15 minutes. Antibody solutions were diluted to a concentration of 1 μg/ml in PCB, and from this point forward, the ELISA was performed similar to the protein ELISA described above.

Ethics

The use of animals in the experiments described in these studies were approved by the Ethical Committee of the Utrecht University and performed according to their regulations.

Hsp70 Localization Studies

The localization of Hsp70 in mycobacteria was studied as follows.

Mycobacteria

The mycobacteria used in these studies are live as well as dead M. a. avium D4, M. a. paratuberculosis 316F (a generous gift from Douwe Bakker, CIDC, Lelystad, The Netherlands), and M. bovis strain 2000-683 (a generous gift from Piet Overduin, RIVM, Bilthoven, The Netherlands). Bacteria were harvested during the mid-logarithmic phase of growth by centrifugation and resuspended in phosphate-buffered saline (PBS) at a concentration of ca 1*10¹⁰ colony-forming units (cfu) per ml as determined spectrophotometrically. M. bovis strain 2000-683 were killed using heat or ethanol (96%) incubation according to established RIVM protocols and, for reasons of comparability, similar methods were used for killing the other mycobacterial species.

Before each assay, bacteria were washed three times with PBS prior to ELISA assays and with PBS supplemented with 1% BSA and 0.01% sodium azide (both from Sigma Aldrich, USA) (FACS buffer) prior to flowcytometry.

Flowcytometry

Suspensions of M. a. avium and M. a. paratuberculosis (both 10¹⁰ bacteria/ml in PBS) were diluted 1:100, washed three times by short centrifuging and resuspended in PBS. These suspensions were diluted 1:100 in FACS buffer and divided in volumes of 100 μl. Antibodies were added in concentrations of approximately 5 μg/ml. After incubation for 25 minutes at room temperature (RT) and three washes with FACS buffer, Alexa633-labeled Goat-anti-mouse (Molecular Probes, USA) was added and incubated for 25 minutes at RT. Following three more washes, up to 10,000 bacterial cells were analyzed using the FACScalibur (Becton-Dickinson, USA).

Immunocapture

The Hsp70-specific monoclonal antibodies (6G1B9) were coupled to Dyna450 magnetic beads according to instructions of the manufacturer (Dynal, Norway), diluted in PBS in concentration of 2×10⁶ beads/ml. Alexa633-labeled M. a. avium and M. a. paratuberculosis were diluted two-fold in PBS after washing and incubated with the Dyna450 beads overnight at RT. After two more washes, immunomagnetic separation was performed according to instructions by the manufacturer (Dynal, Norway), beads with bound bacterial cells were analyzed using the FACScalibur (Becton-Dickinson, USA). Additionally, beads from the described immunomagnetic separation were used for IS900 PCR according to methods published previously.^{[30]}

Electron Microscopy

Bacteria (M. a. paratuberculosis and M. a. avium) were suspended in volumes of 150 μl, washed three times and resuspended in 100 μl block buffer (Roche, Germany). One μg of a 100 μg/ml antibody solution was added and all were incubated top-over-top for 30 minutes. Bacteria were washed three times with PBS containing 0.1% Tween 20 and resuspended in block buffer. Then, protein A coupled to 10 nm gold particles (Aurion, The Netherlands) was added (1:400) and again incubated 30 minutes top-over-top at room temperature. Bacteria were washed as described previously and resuspended in 100 μl block buffer. A 5 μl droplet of bacterial suspension was overlaid with a negative-stain immuno-electron microscopy Ni—or Cu-grid for 15 minutes. The grid was rinsed three times for five minutes on phosphate buffered saline containing 50 mM glycine. Next, the grid was placed on incubation buffer (0.1% acetylated bovine serum albumin in PBS) three times for ten minutes and then washed four times for five minutes using redistilled water. Grids were blotted dry briefly on filter paper, tipped on 5 μl potassium phosphotungstate solution (2%) and blotted completely dry on filter paper. Finally, preparations were viewed using a transmission electron microscope (Philips, The Netherlands).

### REFERENCES

1. Muskens J. et al. Prevalence and regional distribution of paratuberculosis in dairy herds in The Netherlands. Vet. Microbiol. 2000, 77(3-4), p. 253-61.
2. Beard P. M. et al. Paratuberculosis infection of nonruminant wildlife in Scotland. J. Clin. Microbiol. 2001, 39(4), p. 1517-21.
3. Badiola J. J. et al. Possible links between Crohn's Disease and Paratuberculosis. 2000, European Union: Brussel. p. 1-76.
4. Clarke C. J. The pathology and pathogenesis of paratuberculosis in ruminants and other species. J. Comp. Pathol. 1997, 116(3), p. 217-61.
5. Kalis C. H. et al. Use of long-term vaccination with a killed vaccine to prevent fecal shedding of Mycobacterium avium subsp paratuberculosis in dairy herds. Am. J. Vet. Res. 2001, 62(2), p. 270-4.
6. Rothel J. et al. A sandwich enzyme immunoassay for bovine interferon-g and its use for the detection of tuberculosis in cattle. Australian Veterinary Journal, 1990, 67(4), p. 134-137.
7. Milner A. R. et al. The sensitivity and specificity of a modified ELISA for the diagnosis of Johne's disease from a field trial in cattle. Vet. Microbiol. 1990, 25(2-3), p. 193-8.
8. McDonald W. L. et al. Evaluation of diagnostic tests for Johne's disease in young cattle. Aust. Vet. J. 1999, 77(2), p. 113-9.
9. Merkal R. S., K. E. Kopecky and A. B. Larsen. Immunologic mechanisms in bovine paratuberculosis. Am. J. Vet. Res. 1970, 31(3), p. 475-85.
10. Collins M. T. Diagnosis of paratuberculosis. Vet. Clin. North Am. Food. Anim. Pract. 1996, 12(2), p. 357-71.
11. Chiodini R. J. Immunology: resistance to paratuberculosis. Vet. Clin. North Am. Food Anim. Pract. 1996, 12(2), p. 313-43.
12. Sweeney R. W. et al. Interferon-gamma and interleukin 4 gene expression in cows infected with Mycobacterium paratuberculosis. Am. J Vet. Res. 1998, 59(7), p. 842-7.
13. Abbas A., K. Murphy and A. Sher. Functional diversity of helper T-lymphocytes. Nature, 1996, 383, p. 787-93.
14. Estes D. M. and W. C. Brown. Type 1 and type 2 responses in regulation of Ig isotype expression in cattle. Veterinary Immunology and Immunopathology, 2002, 90(1-2), p. 1-10.
15. Koets A. P. et al. Heat-shock protein-specific T-cell responses in various stages of bovine paratuberculosis. Vet. Immunol. Immunopathol. 1999, 70 (1-2), p. 105-15.
16. Wentink G. H. et al. Incidence of paratuberculosis after vaccination against M. paratuberculosis in two infected dairy herds. Zentralbl. Veterinarmed. [B], 1994, 41(7-8), p. 517-22.
17. Koets A. et al. Progressive bovine paratuberculosis is associated with local loss of CD4(+) T-cells, increased frequency of gamma delta T-cells, and related changes in T-cell function. Infect. Immun. 2002, 70 (7), p. 3856-64.
18. Koets A. P. et al. Lewis rats are not susceptible to oral infection with Mycobacterium avium subsp. paratuberculosis. Vet. Microbiol. 2000, 77(3-4), p. 487-495.
19. Young R. Stress proteins and immunology. Annu. Rev. Immunol., 1990, 8, p. 401-20.
20. Zugel U. and S. H. Kaufmann. Role of heat shock proteins in protection from and pathogenesis of infectious diseases. Clin. Microbiol. Rev. 1999, 12(1), p. 19-39.
21. van Eden W. et al. Cloning of the mycobacterial epitope recognized by T-lymphocytes in adjuvant arthritis. Nature, 1988, 331(6152), p. 171-173.
22. Suzue K. and R. A. Young. Heat shock proteins as immunological carriers and vaccines. Exs., 1996, 77, p. 451-65.
23. Munk M. E. and M. Emoto. Functions of T-cell subsets and cytokines in mycobacterial infections. Eur. Respir. J. Suppl. 1995, 20, p. 668s-675s.
24. Bonato V. L. et al. Identification and characterization of protective T-cells in hsp65 DNA-vaccinated and Mycobacterium tuberculosis-infected mice. Infect. Immun. 1998, 66(1), p. 169-75.
25. Lowrie D. et al. Protection against tuberculosis by a plasmid DNA vaccine. Vaccine, 1997, 15(8), p. 834-838.
26. Colston A., I. McConnell and R. Bujdoso. Cloning and expression in Escherichia coli of DNA encoding a 60 kDa stress protein of Mycobacterium paratuberculosis, the causative agent of Johne's disease. Microbiology, 1994, 140, p. 3329-3336.
27. Stevenson K. et al. Complete nucleotide sequence of a gene encoding the 70 kd heat shock protein of Mycobacterium paratuberculosis. Nucleic Acids Res. 1991, 19(16), p. 4552.
28. el-Zaatari F. A. et al. Nucleotide sequence analysis and seroreactivities of the 65K heat shock protein from Mycobacterium paratuberculosis. Clin. Diagn. Lab. Immunol. 1995, 2(6), p. 657-64.
29. Koets A. P. et al. Differential changes in heat shock protein-, lipoarabinomannan-, and purified protein derivative-specific immunoglobulin G1 and G2 isotype responses during bovine Mycobacterium avium subsp. paratuberculosis infection. Infect. Immun. 2001, 69(3), p. 1492-8.
30. Vary P. H. et al. Use of highly specific DNA probes and the polymerase chain reaction to detect Mycobacterium paratuberculosis in Johne's disease. J. Clin. Microbiol. 1990, 28(5), p. 933-7.

1. A method for detecting a Mycobacterium infection in an animal, said method comprising:
providing the animal with a Mycobacterial protein, or functional part, derivative and/or analogue thereof, and
measuring the animal's immune response directed against said Mycobacterium.

2. The method according to claim 1, wherein said Mycobacterial protein, or functional part, derivative and/or analogue thereof, is provided before the Mycobacterium infection.

3. The method according to claim 1, wherein the animal's immune response is measured within twelve months of the Mycobacterium infection.

4. The method according to claim 3, wherein the animal's immune response is measured within 14 days after the animal has been provided with said Mycobacterial protein or functional part, derivative and/or analogue thereof.

5. The method according to claim 1, wherein said measurement of an immune response of the animal comprises a measurement of memory T-cell activation.

6. The method according to claim 1, wherein said measurement of an immune response of the animal comprises a measurement of memory B-cell activation.

7. The method according to claim 1, wherein said Mycobacterium comprises Mycobacterium avium.

8. The method according to claim 7, wherein said Mycobacterium comprises Mycobacterium avium subspecies paratuberculosis.

9. The method according to claim 1, wherein said Mycobacterial protein or functional part, derivative and/or analogue thereof, comprises a surface-associated Mycobacterial protein or functional part, derivative and/or analogue thereof.

10. The method according to claim 9, wherein said Mycobacterial protein or functional part, derivative and/or analogue thereof comprises Mycobacterial heat shock protein 70 or a functional part, derivative and/or analogue thereof.

11. The method according to claim 1, wherein said Mycobacterial protein or functional part, derivative and/or analogue thereof is recombinantly produced.

12. The method according to claim 1, wherein the animal is a ruminant.

13. An isolated peptide comprising a B-cell epitope of a Mycobacterium avium subspecies paratuberculosis heat shock protein 70 or a functional part, derivative and/or analogue thereof.

14. The isolated peptide of claim 13, wherein said B-cell epitope comprises at least a functional part of a sequence as depicted in FIG. 6.

15. The isolated peptide of claim 13, wherein said B-cell epitope comprises at least a functional part of a sequence as depicted in FIG. 7.

16. The isolated peptide of claim 13, wherein said B-cell epitope comprises at least a functional part of a sequence as depicted in FIG. 8.

17. An isolated nucleic acid molecule encoding the isolated peptide of claim 13.

18. An isolated nucleic acid molecule comprising at least a functional part of a sequence as depicted in FIG. 5.

19. The method according to claim 8, wherein the animal is provided with an isolated peptide comprising a B-cell epitope of a Mycobacterium avium subspecies paratuberculosis heat shock protein 70 or a functional part, derivative and/or analogue thereof.

20. A method for detecting a Mycobacterium infection in an animal, said method comprising:
providing the animal with the isolated nucleic acid of claim 17, and
measuring the animal's immune response directed against said Mycobacterium.

21. A diagnostic kit comprising:
the composition of claim 37.

22. The diagnostic kit of claim 21 further comprising:
means for measuring an animal's immune response.

23. The diagnostic kit of claim 21, wherein said Mycobacterium is Mycobacterium avium.

24. The diagnostic kit of claim 23, wherein said Mycobacterial protein comprises a Mycobacterium avium subspecies paratuberculosis protein.

25. The diagnostic kit of claim 24, wherein said Mycobacterial protein comprises a Mycobacterium avium subspecies paratuberculosis heat shock protein 70.

26. A diagnostic kit comprising:
the isolated peptide of claim 13.

27. A primer or probe able to hybridize to the isolated nucleic acid of claim 17.

28. A method of detecting an infection by Mycobacterium in an animal, the method comprising:
utilizing a gene delivery vehicle comprising a nucleic acid encoding a Mycobacterial protein, or a functional part, derivative and/or analogue thereof, to detect Mycobacterial infection in the animal.

29. The method according to claim 28, wherein said Mycobacterium comprises Mycobacterium avium.

30. The method according to claim 29, wherein said Mycobacterium comprises Mycobacterium avium subspecies paratuberculosis.

31. A gene delivery vehicle comprising the isolated nucleic acid of claim 17.

32. A binding molecule selected from the group consisting of an isolated antibody, a functional part thereof, a derivative thereof, and an analogue thereof, said binding molecule able to specifically bind the isolated peptide of claim 13.

33. A method for at least in part treating an infection of a Mycobacterium in an animal, said method comprising:
providing the animal with a Mycobacterial protein, or functional part, derivative and/or analogue thereof.

34. The method according to claim 33, wherein said Mycobacterium comprises Mycobacterium avium.

35. The method according to claim 34, wherein said Mycobacterium comprises Mycobacterium avium subspecies paratuberculosis.

36. The method according to claim 33, wherein said Mycobacterial protein comprises heat shock protein 70.

37. A composition comprising:
an isolated Mycobacterial protein from a Mycobacterium, or a functional part, derivative and/or analogue thereof or an isolated nucleic acid encoding said Mycobacterial protein, or functional part, derivative and/or analogue thereof.

38. The composition of claim 37, wherein said composition is a pharmaceutical composition.

39. The composition of claim 37, wherein said Mycobacterium comprises Mycobacterium avium.

40. The composition of claim 39, wherein said Mycobacterium comprises Mycobacterium avium subspecies paratuberculosis.

41. The composition of claim 40, wherein said Mycobacterial protein comprises a Mycobacterium avium subspecies paratuberculosis heat shock protein 70 or functional part, derivative and/or analogue thereof.

42. A method for at least in part treating a mycobacterial infection of an animal, said method comprising:
providing the animal with the composition of claim 37.

43. An immunogenic composition comprising:
the isolated peptide of claim 13, or a functional part, derivative or analogue thereof.

44. A composition comprising:
an isolated nucleic acid sequence encoding the isolated peptide of claim 13, or a functional part, derivative or analogue thereof.


SEQUENCE LISTING (13 sequences)

SEQ ID NO: 1 - protein, 14 aa, Artificial Sequence
  Epitope of M.a. paratuberculosis Hsp70 protein
ITDAVITVPAYFND

SEQ ID NO: 2 - protein, 14 aa, Artificial Sequence
  Epitope of M.a. paratuberculosis Hsp70 protein
AQAGGPDGAAAGGG

SEQ ID NO: 3 - protein, 14 aa, Artificial Sequence
  Epitope of M.a. paratuberculosis Hsp70 protein
PDGAAAGGGSGSAD

SEQ ID NO: 4 - protein, 14 aa, Artificial Sequence
  Peptide derived from M.a. paratuberculosis Hsp70
AGGGSGSADDVVDA

SEQ ID NO: 5 - protein, 14 aa, Mycobacterium tuberculosis
  SITE (1)..(14): Part of Hsp70 protein
ITDAVITTPAYFND

SEQ ID NO: 6 - protein, 14 aa, Escherichia coli
  SITE (1)..(14): Part of Hsp70 protein
VTEAVITVPAYFND

SEQ ID NO: 7 - protein, 14 aa, Bos taurus
  SITE (1)..(14): Part of Hsc70 protein
VTNAVVTVPAYFND

SEQ ID NO: 8 - DNA, 123 bp, Artificial Sequence
  Nucleic acid sequence derived from M. avium paratuberculosis
cgcggccggcggcgggtccggatccgccgacgatgttgtggacgcggaggtggtcgacgatgaccgggagtccaagtgacacaaggaaatcagaagacggaaggcaacccaatccgacctgca

SEQ ID NO: 9 - protein, 9 aa, Artificial Sequence
  Amino acid sequence derived from M.a. paratuberculosis
PDGAAAGGG

SEQ ID NO: 10 - protein, 557 aa, Mycobacterium a. avium
  SITE (1)..(557): Hsp70
MARAVGIDLGTTNSVVAVLEGGDPVVVANSEGSRTTPSIVAFARNGEVLVGQPAKNQAVTNVDRTIRSVKRHMGTDWSIEIDGKKYTAQEISARVLMKLKRDAEAYLGEDITDAVITVPAYFNDAQRQATKEAGQIAGLNVLRIVNEPTAAALAYGLDKGEKEQTILVFDLGGGTFDVSLLEIGEGVVEVRATSGDNQLGGDDWDDRIVNWLVDKFKGTSGIDLTKDKMAMQRLREAAEKAKIELSSSQSTSINLPYITVDADKNPLFLDEQLTRAEFQRITQDLLDRTRQPFKSVIADAGISVSDIDHVVLVGGSTRMPAVTDLVKELTGGKEPNKGVNPDEKGEVKDVLLLDVTPLSLGIETKGGVMTKLIERNTTIPTKRSETFTTADDNQPSVQIQVYQGEREIAAHNKLLGSFELTGIPPAPRGVPQIEVTFDIDANGIVHVTAKDKGTGKENTIKIQEGSGLSKEEIDRMIVRNQAESLVYQTEKFVKDQREAEGGSKVPEETLSKVDAAIADAKTALGGTDITAIKSAMEKLGQESQALGQAIYEATQAE

SEQ ID NO: 11 - protein, 556 aa, Artificial Sequence
  Consensus sequence
MARAVGIDLGTTNSVVAVLEGGDPVVVANSEGSRTTPSIVAFARNGEVLVGQPAKNQAVTNVDRTIRSVKRHMGTDWSIEIDGKKYTAQEISARVLMKLKRDAEAYLGEDITDAVITVPAYFNDAQRQATKEAGQIAGLNVLRIVNEPTAAALAYGLDKGEKEQTILVFDLGGGTFDVSLLEIGEGVVEVRATSGDNLGGDDWDDRIVNWLVDKFKGTSGIDLTKDKMAMQRLREAAEKAKIELSSSQSTSINLPYITVDADKNPLFLDEQLTRAEFQRITQDLLDRTRQPFKSVIADAGISVSDIDHVVLVGGSTRMPAVTDLVKELTGGKEPNKGVNPDEKGEVKDVLLLDVTPLSLGIETKGGVMTKLIERNTTIPTKRSETFTTADDNQPSVQIQVYQGEREIAAHNKLLGSFELTGIPPAPRGVPQIEVTFDIDANGIVHVTAKDKGTGKENTIKIQEGSGLSKEEIDRMIVRNQAESLVYQTEKFVKDQREAEGGSKVPEETLSKVDAAIADAKTALGGTDITAIKSAMEKLGQESQALGQAIYEATQAE

SEQ ID NO: 12 - protein, 623 aa, Mycobacterium a. paratuberculosis
  SITE (1)..(621): Hsp70
MARAVGIDLGTTNSVVAVLEGGDPVVVANSEGSRTTPSIVAFARNGEVLVGQPAKNQAVTNVDRTIRSVKRHMGTDWSIEIDGKKYTAQEISARVLMKLKRDAEAYLGEDITDAVITVPAYFNDAQRQATKEAGQIAGLNVLRIVNEPTAAALAYGLDKGEKEQTILVFDLGGGTFDVSLLEIGEGVVEVRATSGDNQLGGDDWDDRIVNWLVDKFKGTSGIDLTKDKMAMQRLREAAEKAKIELSSSQSTSINLPYITVDADKNPLFLDEQLTRAEFQRITQDLLDRTRQPFKSVIADAGISVSDIDHVVLVGGSTRMPAVTDLVKELTGGKEPNKGVNPDEVVAVGAALQAGVLKGEVKDVLLLDVTPLSLGIETKGGVMTKLIERNTTIPTKRSETFTTADDNQPSVQIQVYQGEREIAAHNKLLGSFELTGIPPAPRGVPQIEVTFDIDANGIVHVTAKDKGTGKENTIKIQEGSGLSKEEIDRMIKDAEAHAEEDRKRREEADVRNQAESLVYQTEKFVKDQREAEGGSKVPEETLSKVDAAIADAKTALGGTDITAIKSAMEKLGQESQALGQAIYEATQAESAQAGGPDGAAAGGGSGSADDVVDAEVVDDDRESK

SEQ ID NO: 13 - protein, 35 aa, Mycobacterium a. paratuberculosis
  SITE (1)..(35): Part of Hsp70
SAQAGGPDGAAAGGGSGSADDVVDAEVVDDDRESK